(19) United States
(12) Patent Application Publication
(10) Pub. No.: US 2025/0072734 A1
(43) Pub. Date: Mar. 6, 2025

(54) STEERABLE GUIDE WIRE SYSTEMS FOR MEDICAL DEVICES AND RELATED METHODS

(71) Applicants: Boston Scientific Scimed, Inc., Maple Grove, MN (US); Boston Scientific Medical Device Limited, Galway (IE)

(72) Inventors: Deepak Kumar SHARMA, Muzaffarnagar (IN); John Thomas FAVREAU, Charlton, MA (US); Sharath Kumar G, Kanakapura (IN); Juan Pablo ORTIZ GARCIA, Heredia (CR); Brian GAFFNEY, Rutland, MA (US); Shrikant Vasant RAUT, Mumbai (IN)

(73) Assignees: Boston Scientific Scimed, Inc., Maple Grove, MN (US); Boston Scientific Medical Device Limited, Galway (IE)

(21) Appl. No.: 18/817,718

(22) Filed: Aug. 28, 2024

Related U.S. Application Data
(60) Provisional application No. 63/535,777, filed on Aug. 31, 2023.

(51) Int. Cl. A61B 1/01; A61B 1/005; A61B 17/00

(52) U.S. Cl. CPC A61B 1/01; A61B 1/0052; A61B 2017/003

(57) ABSTRACT

A steerable medical device includes a first wire and a second wire. The first wire includes a first portion having a first width and a second portion having a second width. The second width is smaller than the first width. A distal end of the first wire is fixed relative to a distal end the second wire. A proximal end of one or more of the first wire or the second wire is moveable distally or proximally with respect to the other of the first wire or the second wire in order to bend a distal portion of the steerable medical device.

### CROSS-REFERENCE TO RELATED APPLICATIONS

This application claims the benefit of priority to U.S. Provisional Application No. 63/535,777, filed on Aug. 31, 2023, which is incorporated by reference herein in its entirety.

### TECHNICAL FIELD

Aspects of this disclosure generally relate to medical devices and related methods. In particular, aspects of this disclosure relate to steerable guide wires and methods of using same.

### BACKGROUND

Sphincterotomy is a surgical procedure involving the incision or division of a sphincter muscle, commonly performed in the gastrointestinal tract, urinary system, or other areas where sphincter muscles are present. This technique is employed to relieve obstructions, remove stones or foreign bodies, treat strictures, and enable better access for endoscopic procedures. For example, endoscopic retrograde cholangiopancreatography (ERCP) may include performing a sphincterotomy of the papilla of Vater and navigating a guide wire into the bile duct and/or the pancreatic duct. Currently, sphincterotomy is typically performed using rigid or semi-rigid tomes and/or guide wires, which may present challenges in terms of precise navigation and control within the complex anatomical structures.

Existing guide wires lack the necessary flexibility and steerability required to negotiate tortuous paths and tight angles encountered during sphincterotomy procedures. These limitations can lead to increased procedural difficulties, prolonged intervention times, and potential complications. There is a need for an improved guide wire that combines enhanced maneuverability with the necessary structural integrity to ensure optimal performance in a variety of clinical scenarios.

### SUMMARY

Examples of this disclosure relate to, among other things, a steerable medical device including a first wire and a second wire. The first wire may include a first portion having a first width and a second portion having a second width. The second width may be smaller than the first width. A distal end of the first wire may be fixed relative to a distal end the second wire. A proximal end of one or more of the first wire or the second wire may be moveable distally or proximally with respect to the other of the first wire or the second wire in order to bend a distal portion of the steerable medical device.

Any of the devices disclosed herein may include any of the following features, alone or in any combination.

In other embodiments of the steerable medical device, the first portion may have a D-shaped cross-section or a round cross section.

In other embodiments of the steerable medical device, a central longitudinal axis of the first portion is offset from a central longitudinal axis of the second portion.

In other embodiments of the steerable medical device, the second wire may include a third portion having a third width and a fourth portion having a fourth width. The fourth width may be smaller than the third width.

In other embodiments of the steerable medical device, a flat surface of the first portion may face a flat surface of the third portion.

In other embodiments of the steerable medical device, a flat wire may be positioned between the flat surface of the first portion and the flat surface of the third portion.

In other embodiments of the steerable medical device, the first portion, the second portion, and the third portion may be covered by a coating or a sheath. The fourth portion may be uncovered by the coating or the sheath.

In other embodiments of the steerable medical device, the second wire may have a uniform width along an entire length of the second wire.

In other embodiments of the steerable medical device, the first wire may further include a third portion having a third width. The second width may be smaller than the third width.

In other embodiments of the steerable medical device, the second portion may be between the first portion and the third portion.

In other embodiments of the steerable medical device, the first width may be approximately the same as the third width.

In other embodiments of the steerable medical device, the first wire may include a tapered portion tapering between the first width and the second width.

Other embodiments of the steerable medical device may include a handle having an actuator that is configured to move a proximal end of the first wire.

In other embodiments of the steerable medical device, a proximal end of the second wire may be fixed relative to the handle.

In other embodiments of the steerable medical device, the actuator may be configured to transition between a first configuration, in which the actuator is disengaged from the first wire, and a second configuration, in which the actuator is engaged with the first wire.

In yet other embodiments, a steerable medical device may include a first wire; and a second wire. The first wire may include a first portion having a first width and a second portion having a second width. The second width may be smaller than the first width. A distance between the first portion of the first wire and the second wire may be smaller than a distance between the second portion of the first wire and the second wire. One or more of the first wire or the second wire may be moveable distally or proximally with respect to the other of the first wire or the second wire in order to bend a distal portion of the steerable medical device.

Any of the medical devices disclosed herein may include any of the following features, alone or in any combination.

In yet other embodiments of the steerable medical device, the second wire may include a third portion having a third width and a fourth portion having a fourth width. The fourth width may be smaller than the third width. A central longitudinal axis of the third portion may be offset from a central longitudinal axis of the fourth portion.

In still yet other embodiments, a steerable medical device may include a first wire and a second wire. A cross-sectional width of the first wire may vary along a length of the first wire such that a distance between the first wire and the second wire varies. A proximal end of one or more of the first wire or the second wire may be moveable distally or proximally with respect to the other of the first wire or the second wire in order to bend a distal portion of the steerable medical device.

Any of the medical devices disclosed herein may include any of the following features, alone or in any combination.

In still yet other embodiments of the steerable medical device, a cross-sectional width of the second wire may vary along a length of the second wire.

In still yet other embodiments of the steerable medical device, a central longitudinal axis of a first portion of the first wire and a central longitudinal axis of a second portion of the first wire may be concentric and the central longitudinal axis of the first wire is offset from a combined central longitudinal axis of the first wire and the second wire.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1 illustrates a distal portion of a guide wire system for an exemplary medical device, according to aspects of this disclosure.

FIG. 2 illustrates additional details of embodiments of the guide wire system of FIG. 1.

FIG. 3 illustrates additional details of embodiments of the guide wire system of FIG. 1.

FIG. 4 illustrates further details of embodiments of the guide wire system of FIG. 1.

FIG. 5 illustrates further details of embodiments of the guide wire system of FIG. 1.

FIG. 6 illustrates further details of embodiments of the guide wire system of FIG. 1.

FIGS. 7A-7D illustrate another embodiment of a guide wire system.

FIGS. 8 and 9 illustrate yet another embodiment of a guide wire system.

FIG. 10 illustrates yet another embodiment of a guide wire system.

FIG. 11 illustrates yet another embodiment of a guide wire system.

FIGS. 12A and 12B illustrate yet another embodiment of a guide wire system.

FIG. 13 illustrates yet another embodiment of a guide wire system.

FIGS. 14-17 illustrate a handle assembly and specific aspects thereof for controlling a guide wire system such as the guide wire system of FIG. 1.

### DETAILED DESCRIPTION

Examples of this disclosure include devices and methods for a steerable guide wire specifically designed for sphincterotomy procedures. This guide wire incorporates advanced materials, construction techniques, and control mechanisms to enable precise and controlled navigation within the target anatomical structures. The unique design allows for increased maneuverability through tortuous paths, enabling performance of sphincterotomies with increased accuracy, safety, and efficiency. It is to be noted, however, that the scope of subject matter of this application is defined by the features listed in the claims, and not an ability to rectify any particular deficiency.

Reference will now be made in detail to examples of this disclosure described above and illustrated in the accompanying drawings. Wherever possible, the same reference numbers will be used throughout the drawings to refer to the same or like parts.

The terms “proximal” and “distal” are used herein to refer to the relative positions of the components of an exemplary medical device. When used herein, “proximal” refers to a position relatively closer to the exterior of the body of a subject or closer to a user, such as a medical professional, holding or otherwise using the medical device. In contrast, “distal” refers to a position relatively further away from the medical professional or other user holding or otherwise using the medical device, or closer to the interior of the subject's body. As used herein, the terms “comprises,” “comprising,” “has,” “having,” “includes,” “including,” or other variations thereof, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

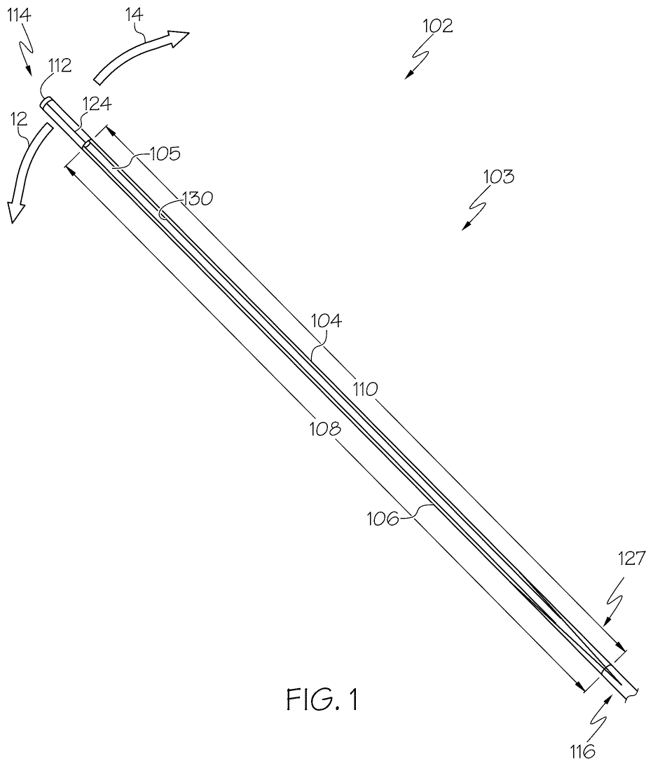

FIG. 1 shows a portion of a steerable guide wire system 102 (or just “guide wire system 102”) having a steerable distal tip section 103 that is formed from a first wire 104 and a second wire 106. Although the term “system” is used herein with respect to guide wire system 102, it will be appreciated that guide wire system 102 may be a single medical device. The first wire 104 and the second wire 106 may be counter-disposed D-shaped wires with an eccentric section 108, 110 in each wire. The eccentric sections 108, 110 may be lengths of the wire with a width that varies along the length and may form a space 105 between the first wire 104 and the second wire 106 that enables relative motion between the wires. The relative motion may be caused by a user (e.g., a physician or other medical device operator) causing one of the first wire 104 or the second wire 106 to move longitudinally with respect to the other, causing the guide wire system 102 to move/articulate left and right (as indicated by arrows 12, 14) within a lumen or other portion of the anatomy of a patient (e.g., gastrointestinal lumen, a urologic lumen, a vein, etc.) The guide wire system is explained in greater detail with respect to FIGS. 1-6.

FIG. 1 shows a distal tip 112 at a distal end 114 of the guide wire system 102. The guide wire system 102 also has a proximal portion 116 at an opposite end of the guide wire system 102 that may be attached to one or more operating assemblies (e.g., a handle assembly as explained in greater detail herein). Motion of the guide wire system 102, including relative motion between the first wire 104 and the second wire 106 may generally be enacted at the proximal end (e.g., a most proximal end of the proximal portion 116) of the guide wire system 102. Aspects of a handle assembly 200, which may be used to control guide wire system 102, are otherwise described herein.

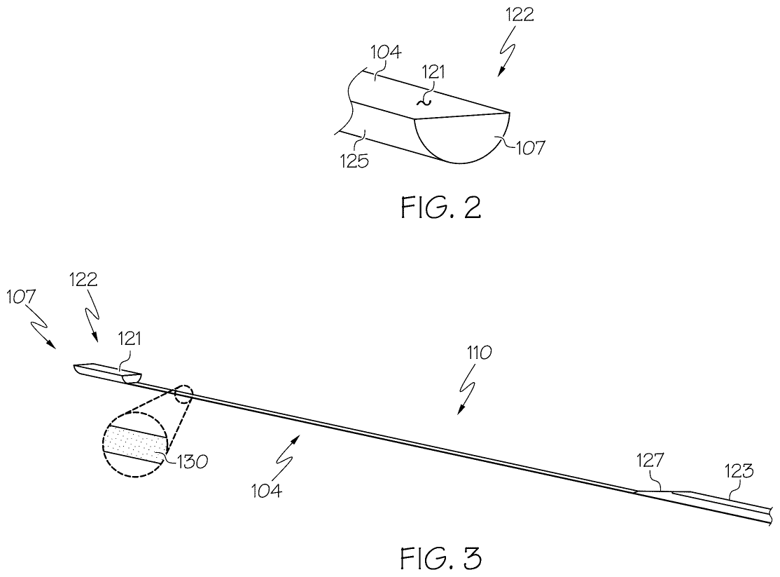

FIGS. 2 and 3 show the first wire 104 in isolation. Any of the features described below, with respect to first wire 104, may also apply to second wire 106. Unless otherwise specified, the same reference numbers apply to first wire 104 as to second wire 106. As mentioned above, the first wire 104 may include generally D-shaped portions (head 122 and proximal portion 123) having a flat face 121 with a rounded outer surface 125. At head 122 and proximal portion 123, a cross-section of wire 104 that is perpendicular to a longitudinal axis of wire 104 may have an approximately D-shape. In some examples, a cross-sectional shape of outer surface 125 may be approximately semi-circular. In other words, head 122 and proximal portion 123 may be similar to a typical round wire divided in half along a center of the round wire.

Wire 104 may include the eccentric section 110 near the distal end 107 of the first wire 104. The eccentric section 110 may end with sufficient length between the distal end of the eccentric section 110 and the distal end 107 of the first wire 104 to form the head 122 at the distal end 107 of the first wire 104. In some embodiments, the head 122 may be between approximately 0.1-3.0 mm in length, between approximately 0.1-10.0 mm in length, or another length. In some embodiments, the eccentric section 110 may be removed by cutting, scraping, grinding, or otherwise removing portions of first wire 104. For example, an entirety of first wire 104 may have a D shape, as described above, prior to formation of eccentric section 110. In some embodiments, the eccentric section can be removed by laser cutting, grinding, or, the wire may be drawn or formed by wire electrical discharge machining into the shape shown in FIG. 3. In other examples, first wire 104, including eccentric section 110, may be formed via additive manufacturing. For example, in some embodiments, one or more portions of the wire 104 may be added via metal 3D printing, laser welding, soldering, or gluing to connect the different sized portions.

The eccentric section 110 may extend along one side of the first wire 104, such as a side of the first wire 104 that is opposite to the face 121 (a portion of the outer surface 125 that is furthest from the face 121). Thus, the eccentric section 110 may be offset from a central longitudinal axis of the first wire 104. In other words, a central longitudinal axis of the eccentric section 110 may be parallel to but not coaxial with the central longitudinal axis of the first wire 104. The flat face 121 of the wire 104 may be cut away from the wire 104, leaving the eccentric section 110.

The eccentric section 110 may have an approximately flat shape in cross-section. For example, the eccentric section 110 may have a ribbon shape. A length of the eccentric section 110 may be approximately 10 mm to approximately 100 mm. The eccentric section 110 may taper proximally to proximal portion 123, forming a tapered portion 127. In some examples, the tapered portion 127 may linearly taper from the eccentric section 110 to the proximal portion 123. The proximal portion 123 may have a same size and shape as the head 122 (e.g., a D-shape). The tapered portion 127 may provide benefits with respect to stress relief for internal stresses within the wire 104 as well as make manufacturing of the wire 104 less complicated. Further, in some embodiments, the flexibility characteristics of the flexible portions of the wire 104 can be gauged based on a size/shape of the tapered portion 127. For example, different taper profiles may result in different bending profiles of wire 104 and may provide for a gradual change in radius of curvature. In some examples, the eccentric section 110 may have a uniform shape between a distal end of the tapered portion 127 and a proximal end of the head 122.

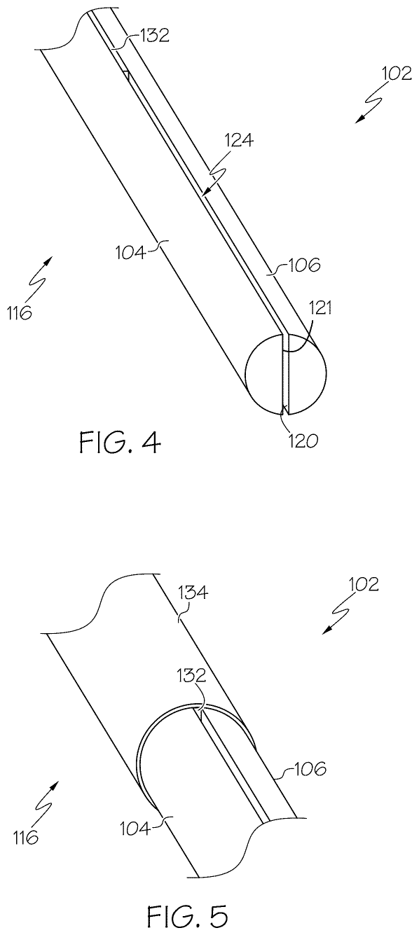

In some embodiments, the second wire 106 may be substantially similar to the first wire 104. When the guide wire system 102 is assembled, the second wire 106 and the first wire 104 may be positioned in a mirrored arrangement across a central longitudinal axis of the guide wire system 102. Referring to FIGS. 1 and 4, the flat faces 120, 121 of the first wire 104 and second wire 106 may face one another at the heads 122 and the proximal portions 123 of the first wire 104 and the second wire 106. The space 105 (a gap) may be formed between the first wire 104 and the second wire 106 along eccentric section 110 of the first wire 104 and an eccentric section 108 of the second wire 106. The space 105 may be formed because the flat faces 120, 121 of first wire 104 and second wire 106 have been removed from the first wire along the eccentric sections 108, 110. The space 105 provides for relative motion between the first wire 104 and the second wire 106 along the eccentric sections 108, 110, which enables the steerable distal tip section 103 to move left and right to steer the guide wire system 102 as shown by arrows 12, 14 (FIG. 1), as discussed below.

As shown in FIG. 3, some embodiments of the guide wire system 102 may include one or more coatings such as the coating 130 on surfaces of the first wire 104 and/or the second wire 106. For example, the eccentric sections 108, 110, the flat face 121 of the first wire 104, the flat face 120 of the second wire 106, or one or more other surfaces may be coated in a coating. For example, the flat face 121 of the first wire 104 and the flat face 120 of the second wire 106 can have a tungsten coating that increases fluoro visibility during a medical procedure using the medical device.

Referring to FIGS. 4-6, further aspects of the guide wire system 102 are shown. FIG. 4 shows a portion of the first wire 104 and the second wire 106 at which the wires may meet at the interface 124 between the flat surfaces at portions of the guide wire system 102 that are proximal of the eccentric sections 108, 110 (e.g., the proximal portion 116). In some embodiments, a flat wire 132 or other feature (e.g., a spacer) can be situated between the first wire 104 and the second wire 106 with the flat faces 120, 121 of each in contact with the flat wire 132. The flat wire 132 may be formed of plastic such as a polyether block amide (PEBA), thermoplastic elastomer (TPE), polytetrafluoroethylene (PTFE), nylon, or other material. In some embodiments, the flat wire 132 can be coated in a coating such as, Nitinol (nickel titanium), MP35N, cobalt-chromium (CoCr), or be a stainless steel wire with a polymer coating that may provide insulation. Sides of the flat wire 132 in contact with the flat surfaces of the first wire 104 and the second wire 106 may have a lower coefficient of friction as compared with the flat surfaces of the first wire 104 and the second wire 106 in direct contact with one another. Additionally, portions of the flat faces of the first wire 104 and the second wire 106 that are not in contact with the flat wire 132 but are nevertheless separated by the space between the first wire 104 and the second wire 106 that the flat wire 132 creates may benefit from additional space between the flat surfaces due to incorporation of the flat wire 132. Accordingly, incorporation of the flat wire 132 between the two portions of the guide wire system 102 may enable better motion between the two wires, thus enabling better control of the steerable distal tip section 103 of the guide wire system 102, improving its maneuverability. Additionally, the flat wire 132 may provide insulation between the two wires, which may be advantageous in embodiments in which one or more of the wires is electrified as explained in greater detail herein.

Referring to FIG. 5, portions of the guide wire system 102 may be wrapped with a sheath 134. For example, portions of the first wire 104 and the second wire 106 may be wrapped in the sheath 134. The sheath 134 can be made from PTFE, polyether block amide (PEBA), silicon, nylon, fluoropolymers (e.g., PFA), other elastomers (ChronoSil, etc.), or other materials. For example, the sheath 134 may include a PTFE shrink-wrap with a silicon coating. In some examples, portions of sheath 134 (e.g., portions formed from PTFE and/or another polymer) may be braided and/or woven The sheath 134 may be heat shrunk onto the guide wire system 102 and may provide electrical and thermal insulation, as well as physical support for keeping the two wires 104, 106 in appropriate positions, improving steerability and performance of the guide wire system 102.

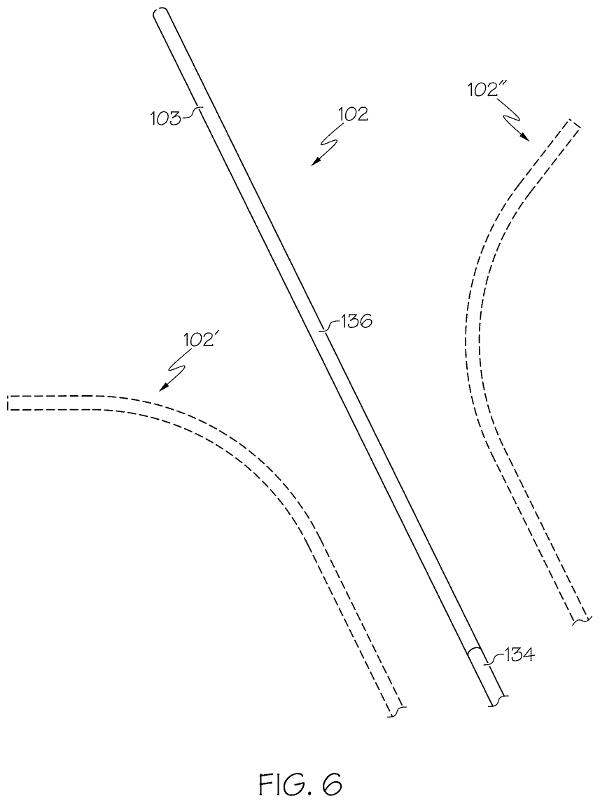

Referring to FIG. 6, the guide wire system 102 is shown with a proximal sheath 134 and a distal sheath 136. The distal sheath 136 may be formed from one or more different materials or otherwise have distinct properties than the proximal sheath 134. The distal sheath 136 may surround at least portions of the steerable distal tip section 103 of the guide wire system 102. The distal sheath 136 can keep the first wire 104 and second wire 106 (which it generally surrounds and hence are not shown in FIG. 6) in a desired position (e.g., may constrain the wires 104, 106 to be within a diameter/width of the sheath 136) as the steerable distal tip section 103 moves left and right to guide the guide wire system 102. In embodiments, the distal sheath 136 may prevent unwanted disjointed bowing between portions of the first wire 104 and portions of the second wire 106 (e.g., between the eccentric sections 108, 110) and may provide thermal and electrical insulation. The sheath 134 and/or the distal sheath 136 may have a variable stiffness along their respective lengths, giving a variable flexibility to the guide wire system 102. In some examples, the sheath 136 may have a relatively lower durometer than the sheath 134, such that the distal tip section 103 may be more flexible than proximal portions of the guide wire system 102. This increased flexibility of the distal tip section 103 may help to allow the distal tip section 103 to articulate (e.g., along the eccentric sections 108, 110).

Referring to FIGS. 1 and 6, in order to articulate the distal tip section 103, one or both of the wires 104, 106 may be pulled proximally or otherwise tensioned. This may change a relative length of the wires 104, 106 within the sheaths 134, 136. For example, in a neutral configuration of the guide wire system 102, the lengths of the wires 104, 106 may be the same or approximately the same. When one of the wires 104, 106 is moved proximally at its proximal end, that wire may be relatively shorter than the other of the wires 104, 106. By way of example, the wire 104 may be moved proximally (tensioned) relative to the wire 106. This may cause the guide wire system 102 to bend in the direction shown by arrow 14 of FIG. 1 (as shown by guide wire system 102″ of FIG. 6). The guide wire system 102 may bend at a location of eccentric sections 108, 110, because the wires 104, 106 may be more flexible at the eccentric sections 108, 110 than at other portions of the wires 104, 106. Similarly, the wire 106 may be pulled proximally (tensioned) relative to the wire 104, so as to cause bending in the direction shown by arrow 12 of FIG. 1 (as shown by guide wire system 102′ of FIG. 6). Alternatively, only one of the wires 104, 106 may be movable. In such an example, the wire 104 or 106 may be movable proximally to articulate/bend the distal tip section 103 in one direction shown by arrow 12 or arrow 14 and may be movable distally to articulate/bend the distal tip section 103 in the other direction shown by arrow 12 or arrow 14. A relative shortening or lengthening of wires 104, 106 with respect to one another may articulate the distal tip section 103. Thus, the guide wire system 102 may be steerable. The steerability of the guide wire system 102 may be beneficial for, for example, gaining access to different ducts of the pancreatico-biliary tree.

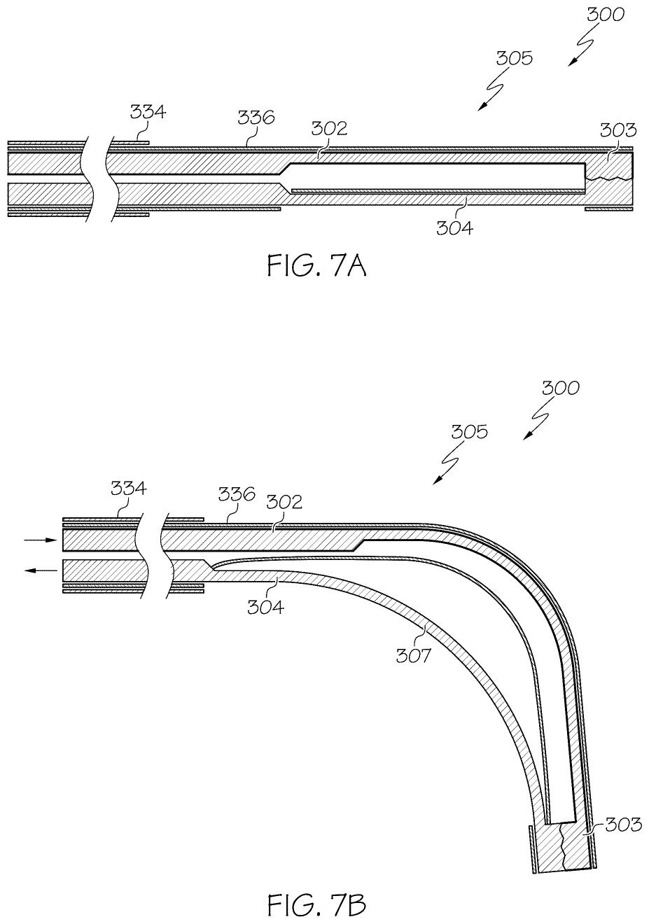

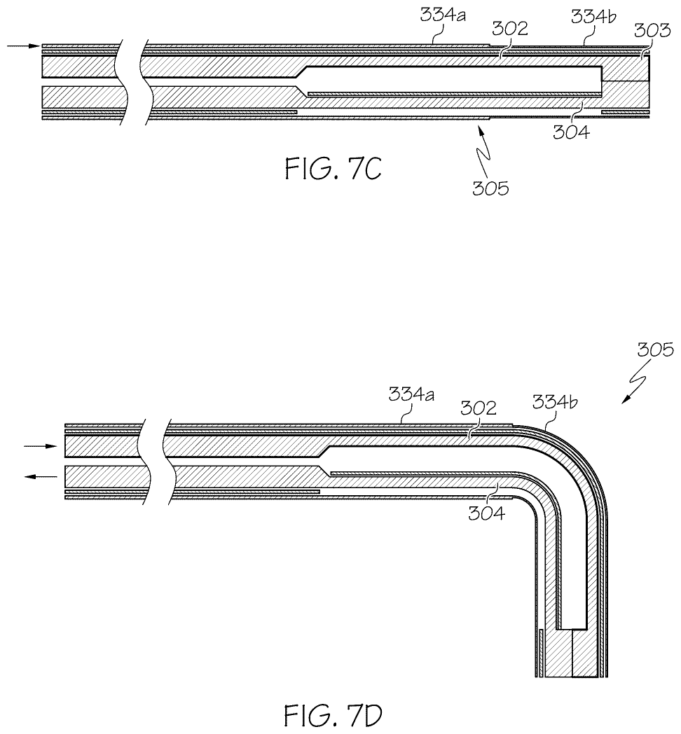

FIGS. 7A-7D show an embodiment of a guide wire system 300 with a first wire 302 and a second wire 304 joined at a distal tip section 303 and having a space 305 between the first wire 302 and the second wire 304. Unless otherwise specifically noted herein, the first wire 302 and the second wire 304 can have any properties, features, and functionality of the other wires mentioned herein. The first wire 302 and the second wire 304 can be surrounded by an outer sheath 334 and an inner sheath 336. Unless otherwise specifically noted herein, the outer sheath 334 and the inner sheath 336 can have any properties, features, and functionality of the other sheaths mentioned herein.

The outer sheath 334 can be moved longitudinally with respect to the length of the first wire 302 and the second wire 304 and portions of the outer sheath 334 can have different characteristics along its length. For example, with particular reference to FIGS. 7C and 7D, the outer sheath 334 can have a high stiffness section 334a and a low stiffness section 334b. Longitudinal movement of the outer sheath 334 can uncover or cover portions of the first wire 302 and the second wire 304 and/or move the sections of variable stiffness with respect to the first wire 302 and the second wire 304. Moving the outer sheath 334, thus, can affect the steerability of the guide wire system 300 by affecting the relative force required to bend the guide wire system at its steerable distal end. For example, moving the outer sheath 334 with respect to the space 305 between the first wire 302 and the second wire 304 can affect movement of the wire. Moving the outer sheath 334 such that it is no longer around the space 305 or such that a length of the outer sheath 334 with a lower stiffness surrounds the space 305 can make it easier for the steerable wire system 300 to move (i.e., can enable the guide wire system 300 to develop relative distal and proximal movement between the first wire 302 and the second wire 304 as explained herein with respect to embodiments of the guide wire system 102). In other embodiments, the durometer o the outer sheath 334 may be sufficiently low (i.e., the outer sheath 334 may be sufficiently pliable) at one or more of its portions such that relative motion between the first wire 302 and the second wire 304 is possible while the outer sheath surrounds some or all of the first wire 302 and the second wire 304 at the space 305.

Still referring to FIGS. 7A-7D, the inner sheath 336 can be an insulating sheath that insulates one or more of the first wire 302 and the second wire 304. The inner sheath 336 may surround all or a portion of the first wire 302 and the second wire 304. As shown in FIGS. 7A and 7B in particular, some portions of the second wire 304 can be exposed from the inner sheath 336, for example, an exposed portion 307. In embodiments, one or more of the first wire 302 and the second wire 304 may be electrified, heated, or otherwise be capable of creating a cut in flesh. With the outer sheath 334 retracted proximally, when a user pushes distally on the first wire 302 or pulls proximally on the second wire 304, the exposed portion 307 may extend away from the first wire 302 due to the relative motion between the wires. The exposed portion 307 of the second wire 304 can act as a tome or knife to cut tissue in a subject.

In order to prevent the second wire 304 from extending away from the first wire 302 when relative longitudinal motion between the two wires is imparted, a user can push the outer sheath 334 distally to surround the first wire 302 and the second wire 304 while relative longitudinal motion between the two wires is developed. This may prevent the second wire 304 from extending away from the first wire 302 and prevent the exposed portion 307 from acting as a tome or knife.

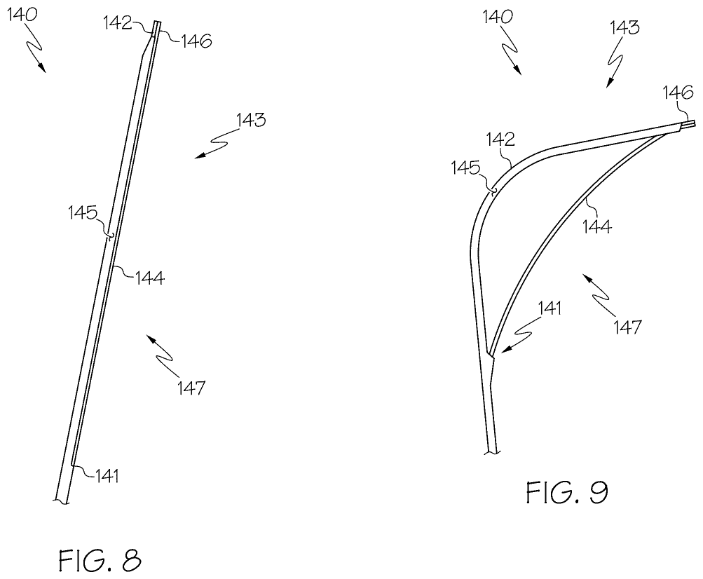

FIGS. 8 and 9 show another embodiment of a guide wire system 140. The guide wire system 140 may include any of the features of the guide wire system 102, unless otherwise specified herein. The guide wire system 140 includes a coated/sheathed wire 142 coated in a coating 145 and an uncoated wire 144 separated by an eccentric portion 147, where the wires 142, 144 are not coupled together. Although the terms “coated” and “uncoated” are used herein, it will be appreciated that portions of the coated wire 142 may be uncoated, and portions of the uncoated wire 144 may be coated, as described below. The wires 142, 144 may have any of the features of the wires 104, 106, discussed above. For example, in some embodiments the wires 142, 144 may have substantially D-shaped heads (similar to the D-shaped head 122 of FIG. 1 and having any of its properties) and proximal portions (having any of the properties of the proximal portions 123), with an eccentric portion (having any of the properties of eccentric sections 108, 110) extending between the heads and proximal portions. The eccentric portion of the wire 142 may not be visible in FIGS. 8 and 9 because it is covered by the coating 145. In other embodiments, the coated wire 142 and/or the uncoated wire 144 may be substantially flat wires.

The uncoated wire 144 may be partially coated along with the coated wire 142 along a portion of a length of uncoated wire 144, and may be exposed from the coating 145 distally of a slit 141 or opening in the coating 145. In some examples, the coating 145 may be disposed on proximal portions of the wires 142, 144 and may join them together as described above for the guide wire system 102. The coating 145 may extend distally along the coated wire 142 but may terminate along a length of the uncoated wire 144 allowing the uncoated wire 144 to move with respect to the coated wire 142. The interface 146 of the wires 142, 144 may be uncoated and the wires 142, 144 may be coupled to one another at the interface, as described above.

The coating 145 on the coated wire 142 may be PTFE, silicon, PEBA, parylene, or another material. In embodiments, the coating can be heat shrunk onto the wire(s). The coated wire 142 and the uncoated wire 144 may be welded together or otherwise joined at the interface 146. Similar to the wires 104, 106, the uncoated wire 144 and the coated wire 142 may be moveable with respect to one another such that one or the other can be pulled in the proximal direction or pushed in the distal direction to steer the guide wire system 140. For example, as shown in FIG. 9, the uncoated wire 144 may be pulled proximally/tensioned, so that it is relatively shortened with respect to the coated wire 142. This may cause a distal end 143 of the guide wire system 140 to bow/articulate in a direction toward a side of the guide wire system 140 having the uncoated wire 144. The uncoated wire 144 may alternatively be moved distally, so that it is relatively lengthened with respect to the coated wire 142. This may cause the distal end 143 to bow/articulate in the opposite direction (toward a side of the guide wire system 140 having the coated wire 142).

Because the uncoated wire 144 is not coupled to the coated wire 142 along the eccentric portion 147, the uncoated wire 144 may separate from the coated wire 142 at the eccentric portion 147, creating a gap between the uncoated wire 144 and the coated wire 142. The bowed, uncoated wire 144 may be similar to a wire of a tome that is used in order to perform a sphincterotomy. In embodiments, the uncoated wire 144 can be electrified (e.g., using a cautery current) and/or heated in order to perform sphincterotomies in a patient.

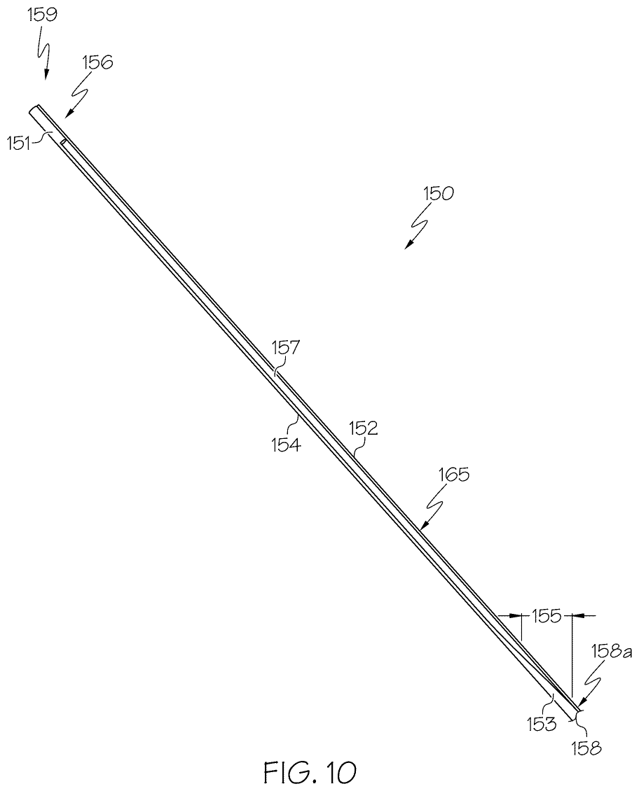

FIG. 10 shows a guide wire system 150 including a first wire 152 and a second wire 154. The first wire 152 may be a flat wire (e.g., a wire that has a uniform diameter or a uniform width along an entire length of the wire) and the second wire 154 may be a wire with similar characteristics as the first wire 104 and/or the second wire 106 of FIG. 1. In some examples, the first wire 152 may have a round cross-sectional shape. In other examples, the first wire 152 may have a flattened cross-sectional shape, so that the first wire 152 has a ribbon-like shape.

The first wire 152 may be coupled to the second wire 154 at an interface 156, which may be at a head 151 (having any of the properties of head 122) of the first wire 152 and a distal end/portion of the second wire 154. A length of a portion of wires 152, 154 that are coupled to one another may be sufficient so as to provide sufficient strength to the guide wire system 150, while allowing for articulation of the guide wire system 150 sufficiently close to a distal end 159 of the guide wire system 150, as described below.

Similarly to the wires 104, 106, the second wire 154 may include a tapered portion 153. The tapered portion 153 may taper from a proximal portion 158 of the second wire 154 to an eccentric portion 165 along a taper distance 155. A space 157 may be formed between the first wire 152 and the second wire 154 along the eccentric portion 165 and the tapered portion 153. The space 157 may be formed because the second wire 154 may extend along the flat face 158a of the proximal portion 158 and the head 151 of the first wire 152, and the second wire 154 may be substantially taught, so that it does not sag within the space 157.

Although not depicted, it will be appreciated that the guide wire system 150 may include any of the sheaths/coatings discussed above. For example, the first wire 152 may include an uncoated portion along eccentric portion 165, similarly to the uncoated wire 144. The guide wire system 150 may further include any of the actuation mechanisms described above. In some examples, a proximal end of the first wire 152 may be moved proximally (pulled or tensioned) or moved distally (pushed or detensioned) in order to articulate the guide wire system 150, as described above for the previous guide wire systems.

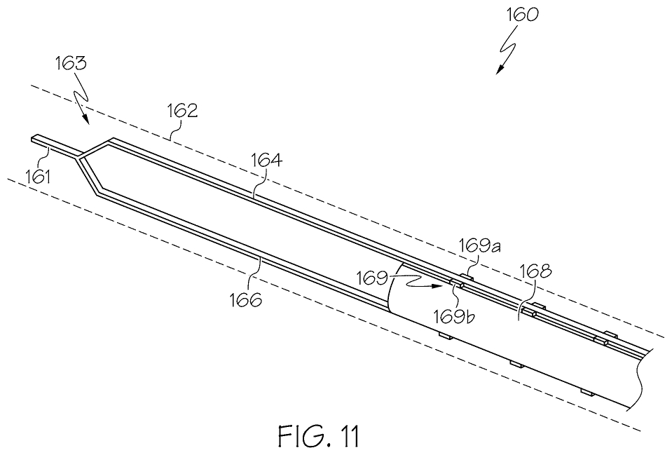

FIG. 11 shows another embodiment of a guide wire system 160. The guide wire system 160 includes a first wire 164 and a second wire 166 separated by a spacer 168. The first wire 164 and the second wire 166 may be flat wires that meet at an interface 163, and at the interface 163 may effectively become a single wire 161. Alternatively, the wires 164, 166 may be a single wire that has a bent/looped distal end in place of interface 163. The first wire 164 and the second wire 166 may include a generally flat cross-section, such that the wires 164, 166 have generally ribbon shapes. In some examples, the wires may 164, 166 may be formed as a material such as Nitinol (nickel titanium). The spacer 168 may be constructed of any suitable material such as, for instance, a copper, plastic, stainless steel, Nitinol coated with silicon, plastic such as PTFE, nylon, or another material.

The spacer 168 may be generally cylindrical and may include a plurality of wire guide mechanisms 169, which may each include wire guides 169a, 169b. The wire guides 169a, 169b may be protrusions that extend radially outward from the spacer 168. The wire guides 169a, 169b may be spaced apart by a distance that is slightly larger than a width of the wires 164, 166. The wires 164, 166 may be slidably received between the wire guides 169a, 169b. The spacer 168 may include two columns of wire guide mechanisms 169—one for each of the respective wires 164, 166. The wire guide mechanisms 169 of each column may be arranged longitudinally along an outer surface of the spacer 168, such that a line drawn along a column of the wire guide mechanisms 169 may form a straight line. Any suitable number of the wire guide mechanisms 169 may be utilized.

The wire guide mechanisms 169 may keep the first wire 164 and the second wire 166 positioned appropriately with respect to one another and the spacer 168. For example, the first wire 164 and the second wire 166 may be kept at generally opposite sides of the spacer 168 (diametrically opposed to one another). Because the wire guide mechanisms 169 may be arranged longitudinally along the spacer 168, the wires 164, 166 may also be positioned longitudinally along the spacer 168. The wire guides 169a, 169b can be positioned along any portion of the length of the spacer 168. The spacer 168 may have a distal end that is far enough away from the interface 163 such that the first wire 164 and the second wire 166 may move longitudinally with respect to one another, providing a bending capability to the two wires and hence a steering capability to the guide wire system 160.

The wires 164, 166 may be moved proximally and/or distally in order to articulate the guide wire system 160. In some examples, both of the wires 164, 166 may be actively moved; in other examples, only one of the wires 164, 166 may be actively moved. Because of the spacer 168, the wires 164, 166 may be offset from a central longitudinal axis of the guide wire system 160 (which may be a central longitudinal axis of the spacer 168). Thus, pushing/pulling on the wires 164, 166 may generate a torque that deflects/articulates the guide wire system 160. In some example, the guide wire system 160 may be bendable distal to a distal end of the spacer 168. In other examples, the spacer 168 may be sufficiently flexible (and/or may include features such as slits or the like), such that the guide wire system 160 may be steerable at portions of the guide wire system 160 having the spacer 168.

In some embodiments, the spacer 168 may be longitudinally moveable with respect to the interface 163 (that is, the longitudinal distance between a distal end of the spacer and the interface 163 can increase or decrease) to change a bending radius of the guide wire system 160. A user may push or pull the spacer 168 and/or jointly push or pull the first wire 164 and/or the second wire 166 to change the distance between the interface 163 and an end of the spacer 168. When a distance between the spacer 168 and the interface 163 is relatively smaller, a bending radius of the guide wire system 160 may be relatively smaller. When a distance between the spacer 168 and the interface is relatively larger, a bending radius of the guide wire system 160 may be relatively larger.

A sheath 162 may surround all or a portion of the other components of the guide wire system 160. The sheath 162 may have a variable stiffness along its length, which variable stiffness can add a dimension of control to the location and degree of bend of the guide wire system 160. For example, a portion of the sheath with an increased stiffness may be less bendable than a portion of the sheath 162 with a reduced stiffness. Portions of increased or decreased relative stiffness can be moved forward or backward or joined together along a length of the sheath 162 to control the capability of motion of the guide wire system 160.

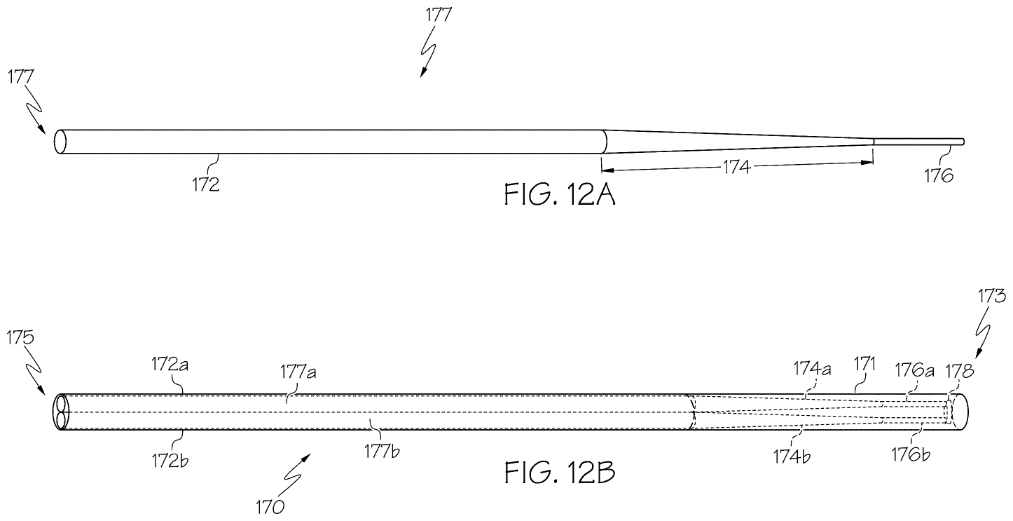

FIGS. 12A and 12B show aspects of yet another embodiment of a guide wire system 170. The guide wire system 170 is formed from a first wire 172a and a second wire 172b that are surrounded by a sheath 171 and joined at a common end 178. The common end 178 can be a welded end piece that is welded at a distal end 173 of the guide wire system 170, joining both the first guide wire 172a and the second guide wire 172b. The distal end 173 may be opposite a proximal end 175 of the guide wire system 170.

FIG. 12B shows a wire 172. Wire 172 may be used as the first wire 172a and as the second wire 172b. The wire 172 may have a tapering portion 174 that tapers towards an end portion 176 at a distal end of the wire 172. The end portion 176, distal of the tapering portion 174, may have an approximately uniform diameter/width. A proximal portion 177 of the wire 172, proximal of the tapering portion 174, may also have an approximately uniform diameter/width. The diameter/width of the proximal portion 177 may be larger than the diameter/width of the end portion 176, and the tapering portion 174 may taper from the larger width of the proximal portion 177 to the smaller width of the end portion 176. The tapering portion 174 may taper symmetrically, such that a central longitudinal axis of the tapering portion and the end portion 176 is coaxial with a central longitudinal axis of the proximal portion 177. Alternatively, the tapering portion 174 may taper eccentrically, such that an angle of the taper of the tapering portion 174 differs around a circumference of the tapering portion 174.

The first wire 172a and the second wire 172b may include tapering portions 174a, 174b that correspond to the tapering portions 174. The first wire 172a and the second wire 172b may similarly include end portions 176a, 176b and proximal portions 177a, 177b that correspond to the end portion 176 and the proximal portion 177, respectively. The proximal portions 177a, 177b may be adjacent to one another, and the tapering portions 174a, 174b and end portions 176a, 176b may be spaced apart from one another, such that there is a gap between portions of the wires 172a, 172b distal of proximal ends of the tapering portions 174a, 174b. In embodiments, one or more of the various portions may be concentric with one or more of the others. The common end 178 may be a piece that extends approximately perpendicularly to a central longitudinal axis of the guide wire system 170, such that it joins distalmost ends of the end portions 176a, 176b.

Thus, the first wire 172a and the second wire 172b may each be offset (e.g., parallel to but not coaxial with) a central longitudinal axis of the guide wire system 170. The space between the first wire 172a and the second wire 172b may be such that they can move with respect to one another, controlling the motion of the guide wire system 170. For example, the first wire 172a and/or the second wire 172b may be moved proximally or distally (tensioned or detensioned) in order to articulate the guide wire system 170, similarly to the other guide wire systems described above.

The sheath 171 may have a variable stiffness along its length such that portions of the sheath 171 are more flexible than others. Hence, the sheath 171 can modulate the flexibility of the guide wire system 170 as it is used to steer left or right in the anatomy of subject.

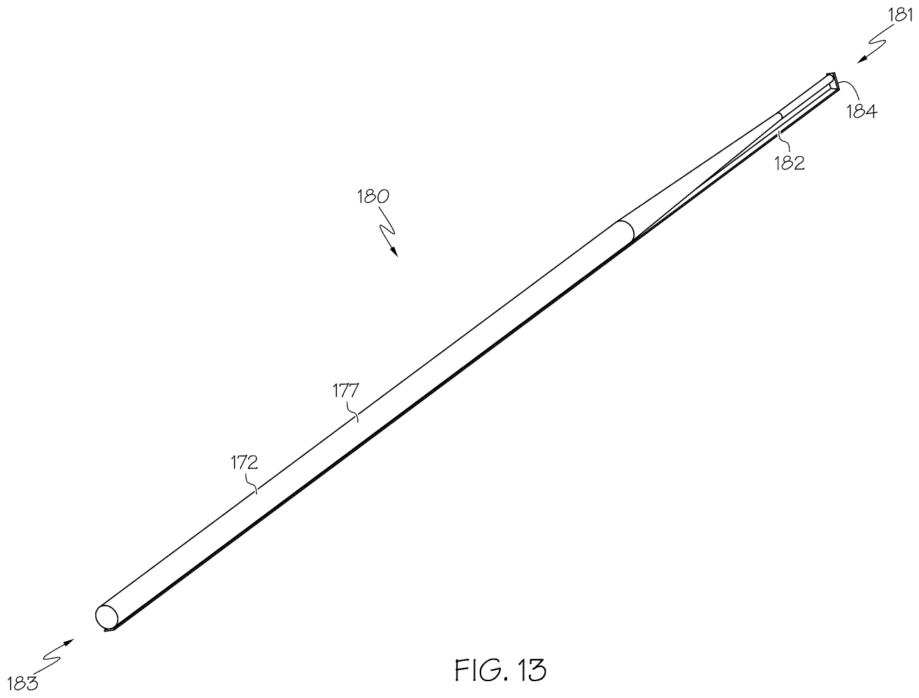

FIG. 13 shows a guide wire system 180 that includes a wire 172 (having any of the properties of wire 172 of FIG. 12B) and a flat wire 182. The flat wire 182 may have any of the properties of the flat wires 142, 144 described above. The flat wire 182 may have a flat cross-sectional shape, such that the wire 182 has a ribbon shape. The wire 172 and the flat wire 182 may be coupled by an end piece 184 having any of the properties of the common end 178. The end piece 184 may be welded or otherwise coupled to the wire 172 and the flat wire 182 at a distal end 181 of the guide wire system 180 that is opposite a proximal end 183 of the guide wire system 180. Although not shown, a sheath (having any of the properties of the sheath 171) may surround the wire 172 and the flat wire 182.

Along the proximal portion 177 of the wire 172, the flat wire 182 may be adjacent to (e.g., in contact with) the wire 172. Along the tapering portion 174 and the end portion 176, the flat wire 182 may be spaced apart from the wire 172. The end piece 184 may maintain the space between the flat wire and the tapering portion 174 and the end portion 176 of the wire 172. The flat wire 182 may be sufficiently taught and/or rigid, such that the space between the wire 172 and the flat wire 182 is maintained.

In operation, the wire 172 may be moved proximally (pulled) and/or distally (pushed), in order to shorten or lengthen a portion of the wire 172 within the sheath (not shown) relative to the flat wire 182. Thus, the guide wire system 180 may articulate, similarly to the guide wire system 150, described above.

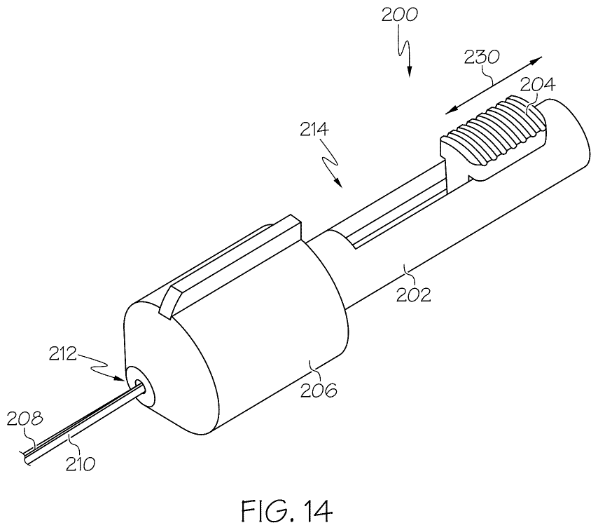

FIGS. 14-17 show a handle assembly 200 for use with any of the various guide wire systems described herein. Although the handle assembly 200 shown in FIG. 14-17 is described with respect to a guide wire system with two opposing D-shaped wires such as the guide wire system 102 described with respect to FIGS. 1-6, the principals of operation described with respect to the handle assembly 200 apply to the other embodiments described herein.

FIG. 14 shows the handle assembly 200 including a handle body 202. The handle assembly 200 may include an actuator such as a slider or knob 204 that is slideably connected to the handle body 202 inside a slot 214 of the handle body 202 and is configured to transition between one or more configurations. The handle body 202 may be threadably connected to a cap 206. D-shaped wires 208, 210 extend distally out of a channel 212 in the cap 206. The channel 212 in the cap 206 may be in communication with the lumen 213 in the handle body 202. Although not shown, a sheath may surround the wires 208, 210, as described above.

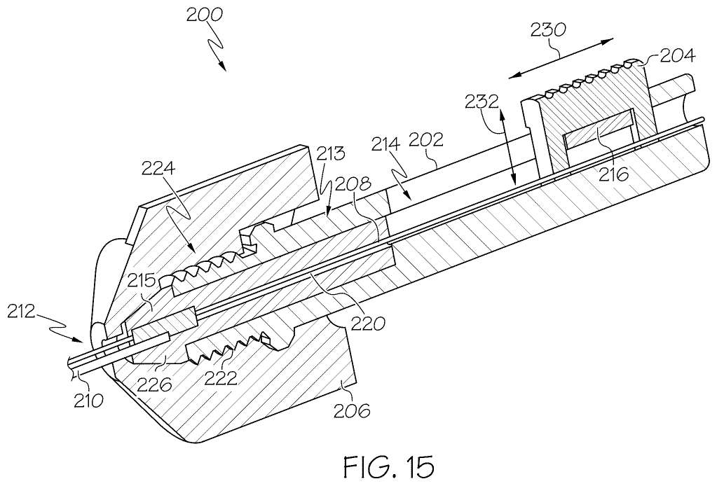

FIG. 15 shows a cross-sectional view of the handle body 202. The cap 206 and the handle body 202 may be joined at a threaded portion 224 of the cap 206 and a threaded portion 222 of the handle body 202. A collet 226 may be positioned within a lumen 213 of the handle body 202, such that a distal portion 215 of the collet 226 extends through a distal opening of the lumen 213 and distal to a distalmost end of the handle body 202. The distal portion 215 of the collet may be received within the cap 206.

The channel 212 may be in communication with a channel 220 of the collet 226. The channel 220 and the channel 212 may be coaxial. One of the D-shaped wires 208 may extend through the channel 212 and the channel 220 and be moveable in a distal and proximal direction with respect to the collet 226 based on movement of the knob 204, as explained in greater detail herein. The other of the D-shaped wires 210 may extend through the channel 212 and may include a hypotube welded or crimped thereto. When the cap 206 is screwed onto the handle body 202, the cap 206 may compress the collet 226, thus gripping the hypotube around the wire 210 and fixing the wire 210 relative to the collet 226. Alternatively, the wire 210 may be welded to a portion of the collet 226 or otherwise coupled to the handle assembly 200 such that it is not moveable in a distal and proximal direction with respect to the collet 226/handle body 202.

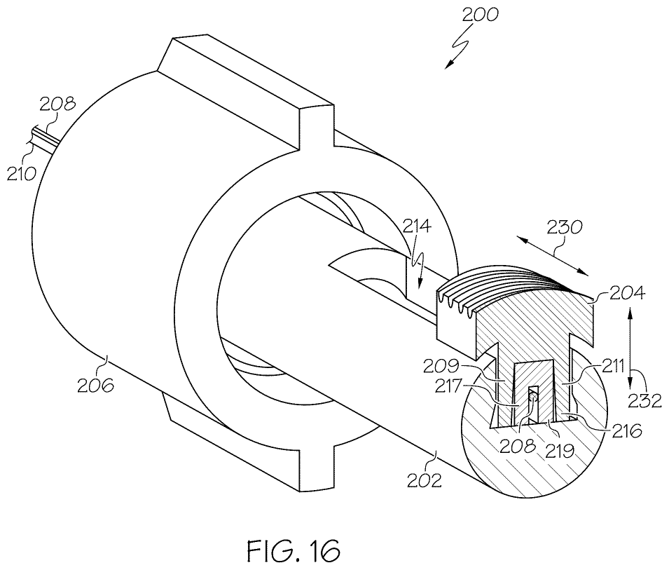

As shown in FIG. 15, the knob 204 can move back and forth (proximally and distally) in the slot 214 as shown by arrow 230 and up and down (radially inward and outward) within the slot 214 as shown by arrow 232 to disengage and engage the D-shaped wire 208, as described below. Referring to FIGS. 15 and 16, the knob 204 includes a gripper 216. The gripper 216 may have two walls 209, 211, which may taper outward, as shown in FIG. 16. Knob 204 may include two walls 217, 219, which may interact with the walls 209, 211 of the gripper 216 as the knob 204 is pushed downward/radially inward, resulting in a wedge action on the gripper 216. The walls 209, 211 of the knob 204 may push the walls 217, 219 toward one another, so that they grip onto the wire 208.

When the knob 204 is moved to an active control position by a user (e.g., pressed down in the downward/radially inward direction of the arrow 232), the gripper 216 and knob 204 may surround a proximal portion of the moveable D-shaped wire 208 with a friction fit. Gripper 216 may not frictionally engage the D-shaped wire 208 the D-shaped wire 210 until a user presses down on the knob 204. Thus, any proximal/distal movement of the knob 204 without pushing down on knob 204 may not engage wire 208 (the knob 204 may move relative to the wire 208 when the knob 204 is not pushed downward). Subsequently to pressing downward on the knob 204, the knob 204 may be moved distally and proximally with respect to the handle assembly 200, thereby moving the D-shaped wire 208 distally and proximally with respect to the handle body 202.

Referring to FIGS. 15 and 16, because the D-shaped wire 210 is fixed to the collet 226 or another portion of the handle assembly 200, its motion in the distal and proximal directions is inhibited and, in embodiments, the D-shaped wire 210 does not move distally and proximally as the D-shaped wire 208 does. Hence, movement of the knob 204 generates relative distal and proximal movement between the D-shaped wire 208 and the D-shaped wire 210. This allows the guide wire system 102 to articulate (e.g., left and right as shown by arrow 12 and arrow 14 of FIG. 1) based on the direction in which the user moves the knob 204. More specifically, if the user moves the D-shaped wire 208 distally with respect to the D-shaped wire 210 (i.e., moves the knob 204 distally toward the cap 206), the two-wire system will tend to curl/bend away from the side of a shaft having the D-shaped wire 208. Conversely, if the user moves the D-shaped wire 208 proximally with respect to the D-shaped wire 210 (i.e., moves the knob 204 away from the cap 206), the two-wire system will tend to curl/bend towards the side of the device having the D-shaped wire 208.

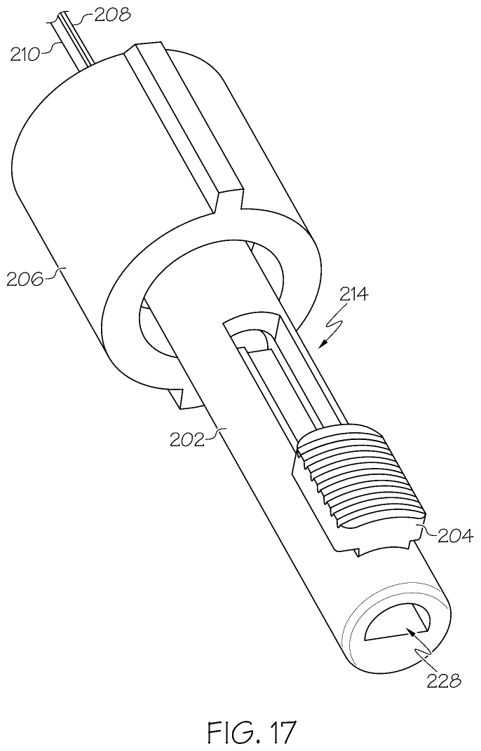

FIG. 17 shows a port 228 in a proximal end of the handle body 202. The port 228 can be used for, for example, loading one or more wires into the handle assembly 200. For example, one or both of the D-shaped wires 208, 210 can be loaded into the port 228 and pushed through the channel 220 in the collet 226 and the channel 212 in the cap 206. Alternatively, the port 228 may be used for delivering contrast or other agents.

Embodiments of the present disclosure may be applicable to various and different medical or non-medical procedures. In addition, certain aspects of the aforementioned embodiments may be selectively used in collaboration, or removed, during practice, without departing from the scope of the disclosure.

While principles of this disclosure are described herein with reference to illustrative aspects for particular applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

We claim:

1. A steerable medical device comprising:
a first wire; and
a second wire;
wherein the first wire includes a first portion having a first width and a second portion having a second width, wherein the second width is smaller than the first width,
wherein a distal end of the first wire is fixed relative to a distal end the second wire, and
wherein a proximal end of one or more of the first wire or the second wire is moveable distally or proximally with respect to the other of the first wire or the second wire in order to bend a distal portion of the steerable medical device.

2. The steerable medical device of claim 1, wherein the first portion has a D-shaped cross-section or a round cross-section.

3. The steerable medical device of claim 1, wherein a central longitudinal axis of the first portion is offset from a central longitudinal axis of the second portion.

4. The steerable medical device of claim 1, wherein the second wire includes a third portion having a third width and a fourth portion having a fourth width, wherein the fourth width is smaller than the third width.

5. The steerable medical device of claim 4, wherein a flat surface of the first portion faces a flat surface of the third portion.

6. The steerable medical device of claim 5, wherein a flat wire is positioned between the flat surface of the first portion and the flat surface of the third portion.

7. The steerable medical device of claim 4, wherein the first portion, the second portion, and the third portion are covered by a coating or a sheath, and wherein the fourth portion is uncovered by the coating or the sheath.

8. The steerable medical device of claim 1, wherein the second wire has a uniform width along an entire length of the second wire.

9. The steerable medical device of claim 1, wherein the first wire further includes a third portion having a third width, and wherein the second width is smaller than the third width.

10. The steerable medical device of claim 9, wherein the second portion is between the first portion and the third portion.

11. The steerable medical device of claim 9, wherein the first width is approximately the same as the third width.

12. The steerable medical device of claim 1, wherein the first wire includes a tapered portion tapering between the first width and the second width.

13. The steerable medical device of claim 1, further comprising a handle having an actuator that is configured to move a proximal end of the first wire.

14. The steerable medical device of claim 13, wherein a proximal end of the second wire is fixed relative to the handle.

15. The steerable medical device of claim 13, wherein the actuator is configured to transition between a first configuration, in which the actuator is disengaged from the first wire and a second configuration, in which the actuator is engaged with the first wire.

16. A steerable medical device comprising:
a first wire; and
a second wire;
wherein the first wire includes a first portion having a first width and a second portion having a second width, wherein the second width is smaller than the first width,
wherein a distance between the first portion of the first wire and the second wire is smaller than a distance between the second portion of the first wire and the second wire, and
wherein one or more of the first wire or the second wire is moveable distally or proximally with respect to the other of the first wire or the second wire in order to bend a distal portion of the steerable medical device.

17. The steerable medical device of claim 16, wherein the second wire includes a third portion having a third width and a fourth portion having a fourth width, wherein the fourth width is smaller than the third width, and a central longitudinal axis of the third portion is offset from a central longitudinal axis of the fourth portion.

18. A steerable medical device comprising:
a first wire; and
a second wire;
wherein a cross-sectional width of the first wire varies along a length of the first wire such that a distance between the first wire and the second wire varies, and
wherein a proximal end of one or more of the first wire or the second wire is moveable distally or proximally with respect to the other of the first wire or the second wire in order to bend a distal portion of the steerable medical device.

19. The steerable medical device of claim 18, wherein a cross-sectional width of the second wire varies along a length of the second wire.

20. The steerable medical device of claim 18, wherein a central longitudinal axis of a first portion of the first wire and a central longitudinal axis of a second portion of the first wire are concentric and the central longitudinal axis of the first wire is offset from a combined central longitudinal axis of the first wire and the second wire.
